(19) United States
(12) Patent Application Publication
(10) Pub. No.: US 2024/0391868 A1
(43) Pub. Date: Nov. 28, 2024

(54) SULFONATE COMPOUNDS

(71) Applicants: Karl Franzens Universität Graz, Graz (AT); Rijksuniversiteit Groningen, CP Groningen (NL)

(72) Inventors: Katalin BARTA WEISSERT, Graz (AT); Markus HOCHEGGER, Graz (AT); Fridrich BÁLINT, AG Groningen (NL)

(21) Appl. No.: 18/693,875

(22) PCT Filed: Sep. 21, 2022
(86) PCT No.: PCT/EP2022/076252

§ 371 Date: Mar. 20, 2024

(30) Foreign Application Priority Data
Sep. 21, 2021 (EP) 21198127.9

(51) Int. Cl. C07C 309/24; C11D 1/00

(52) U.S. Cl. CPC C07C 309/24; C11D 1/002

(57) ABSTRACT

Sulfonate compounds according to formula (I) or (II), a preparation method, and their use as surfactants are provided.

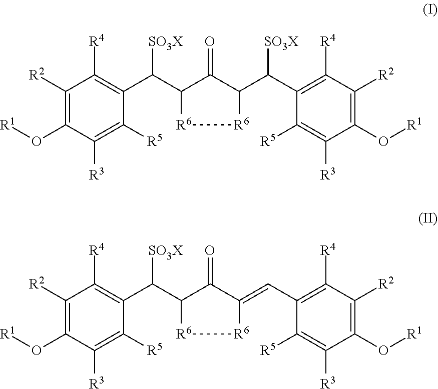

Each R¹ is selected from hydrocarbon radicals having 4 to 26 C atoms and optionally at least one O or S atom; R² to R⁵ are each independently selected from hydrogen and hydrocarbon radicals having 1 to 26 C atoms and optionally at least one O or S atom; each R⁶ is independently selected from hydrogen and hydrocarbon radicals having 1 to 6 carbon atoms, optionally the two radicals R⁶ may be connected, as indicated by the dashed line, to form a five-or six-membered ring containing the carbonyl carbon atom; and each X is independently selected from mono-or polyvalent cations Xⁿ⁺, wherein n is ≥1, including H⁺, wherein, in formula (I), optionally both X together may represent a polyvalent cation.

The present invention relates to novel sulfonate compounds, a method for preparing them and their use as surfactants.

### BACKGROUND OF THE INVENTION

Lignin is an abundant aromatic biopolymer, the structure of which is based primarily on three substituted phenols, so-called monolignols (p-coumaryl, coniferyl and sinapyl alcohol), which are characterized by a variety of different C—O and C—C bonds forming an amorphous 3-dimensional structure. Different methods have been developed to enable the catalytic degradation of lignin by means of depolymerization in order to obtain industrially usable monomolecular phenol and/or benzaldehyde derivatives. In addition to monomers, the products of these depolymerization processes may also contain phenolic di-, tri-and oligomers.

Only recently, various methods have been developed through which selective depolymerization of lignin has become possible, mainly involving reductive or oxidative reaction strategies. The former usually yield phenol, guaiacol or syringol derivatives with aliphatic radicals being typically one to three carbon atoms in length and bearing alcohol, aldehyde, ester and/or ketone functionalities. The latter, on the other hand, typically provide aromatic aldehydes such as vanillin or syringaldehyde or similarly functionalized guaiacol and syringol derivatives. The main products of such depolymerization processes include guaiacol and syringol or vanillin and syringaldehyde, respectively, each of which often have one or more alkyl and/or alkoxy substituents on the aromatic ring.

All of the lignin degradation products mentioned represent valuable biologically based resources from which a number of different products have been produced in recent years. According to the inventors' research, there are hardly any surfactants, although, also in this area, there is a need for products that can be synthesized based on non-edible, renewable raw materials.

Against this background, the aim of the invention was the synthesis of novel chemical compounds by functionalizing these products of lignin degradation and similar compounds, preferably in an environmentally friendly manner, which are particularly suitable for use as surfactants.

### SUMMARY OF THE INVENTION

The present invention achieves this goal in a first aspect by providing novel sulfonate compounds according to formula (I) or (II):

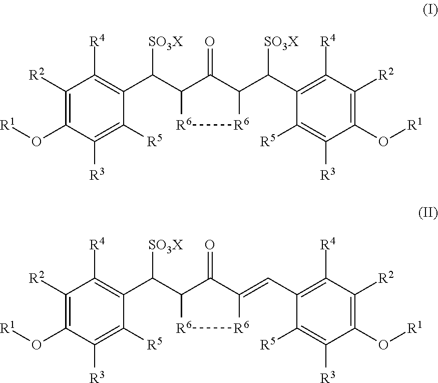

wherein
each R¹ is selected from linear, branched or cyclic hydrocarbon radicals having 4 to 26 carbon atoms, wherein optionally at least one carbon atom may be replaced by an oxygen or sulfur atom;
R² to R⁵ are each independently selected from hydrogen and linear, branched or cyclic hydrocarbon radicals having 1 to 26 carbon atoms, wherein optionally at least one carbon atom may be replaced by an oxygen or sulfur atom;
each R⁶ is independently selected from hydrogen and saturated hydrocarbon radicals having 1 to 6 carbon atoms, wherein optionally the two radicals R⁶ may be connected, as indicated by the dashed line, to form a five-or six-membered ring comprising the carbonyl carbon atom; and
each X is independently selected from mono-or polyvalent cations Xⁿ⁺, wherein n is ≥1, including H⁺, wherein, in formula (I), optionally both X together may represent a polyvalent cation.

As the inventors have found, such disulfonate compounds according to formula (I) or corresponding monosulfonates according to formula (II) can be produced in a relatively simple and environmentally friendly manner from readily available lignin degradation products and are excellently suited as surfactants. Due to the high hydrophilicity of the sulfonate group(s) and the hydrophobicity of the aromatic(s), even a single-digit number of carbon atoms in the radicals R¹ to R⁵ is sufficient to impart the compounds the required amphiphilicity.

However, the number of carbon atoms in the radicals R¹ to R⁵ is preferably at least 9 carbon atoms. This is particularly preferable with regard to hydrophobicity if two sulfonate groups —SO₃X are bonded to the central pentanone in the compounds according to formula (I). But the fact that the main products of the depolymerization of lignin, as mentioned at the beginning, include derivatives of vanillin and syringaldehyde, which often have one or two additional lower alkyl and/or lower alkoxy substituents, simplifies the synthesis of inventive sulfonate compounds having at least 9 carbon atoms in the radicals R¹ to R⁵, since R² to R⁴ already contain several carbon atoms. Consequently, in the preparation method according to the invention, only the free phenolic OH group of such a vanillin or syringaldehyde derivative needs to be etherified with an easily available and biodegradable fatty alkyl radical.

Since, on the one hand, fatty alcohols naturally occur in both saturated and unsaturated form, i.e. with one or more C═C double bonds, and on the other hand, as mentioned at the beginning, the lignin degradation products may also have more than one aromatic, but also non-aromatic rings (e.g. dioxolane) as substituents, the definition of R¹ to R⁵ according to the present invention includes both saturated and unsaturated as well as cyclic radicals.

The fact that, in addition to the disulfonate compounds according to formula (I), monosulfonates according to formula (II) are also included in the present invention is due to the sulfonation by addition of hydrogen sulfite in step 3) of the inventive method, which will be explained in more detail in relation to this second aspect of the invention and will be proven by the examples thereinafter.

In general, it should be noted that “sulfonate” is to be understood herein not only as salts of the sulfonic acid group(s), but also as the respective free acid, unless the context requires otherwise. This is also documented by the above definition of the counterion X, which explicitly also includes H⁺. This is because when the novel compounds are used as surfactants according to the third aspect of the invention, the respective free acids also become ionized in an aqueous environment, thereby forming sulfonate groups in situ.

The lower and upper limits for the number of carbon atoms in the radicals R¹ to R⁵ refer to the preferred use of fatty alkyl radicals for the etherification of free phenolic OH groups in the starting products, for the chain length of which literature specifies from 4 to 6 as the lower limit and from 22 to 26 as the upper limit. According to the invention, a maximum length of 18 carbon atoms is preferred for the fatty alkyl radicals introduced in the synthesis method by means of etherification, and a maximum length of 4 carbon atoms is preferred for the alkyl or alkoxy, or optionally also alkylthio, radicals already bound to the aromatics in the starting material, which is particularly true for the radicals R² and R³ in the ortho position to the phenolic OH group.

The option that some carbon atoms may be replaced by oxygen or sulfur also refers primarily to the substitution pattern of the starting compounds, which are preferably obtained by lignin depolymerization and which, as mentioned at the beginning, may comprise various oxygen-containing functionalities, but sometimes also sulfur analogues thereof. Other heteroatoms, such as halogens or nitrogen, are hardly present in such compounds. Therefore, for the purposes of the present invention, heteroatoms other than oxygen and sulfur need not be considered.

Therefore, in some preferred embodiments of the invention, R¹ is C₆-C₂₂ alkyl, more preferably C₈-C₁₈ alkyl. Alternatively or additionally, in some preferred embodiments, R² and R³ are selected from hydrogen, C₁-C₂₂ alkyl, and C₁-C₂₂ alkoxy, more preferably from hydrogen, C₁-C₄ alkyl, and C₁-C₄ alkoxy; for example, one of R² and R³ is hydrogen and the other is methoxy or both radicals are methoxy. Alternatively or additionally, in some preferred embodiments, R⁴ and R⁵ are selected from hydrogen, methyl, and methoxy. Alternatively or additionally, in some preferred embodiments, the radicals R⁶ are selected from hydrogen, methyl, and ethyl or are connected to one another to form a five-or six-membered ring comprising the carbonyl carbon. Alternatively or additionally, in some preferred embodiments, each X represents H⁺, Na⁺, K⁺, NH₄⁺, or an organic ammonium ion.

In particularly preferred embodiments, with regard to the synthesis method, both radicals of R¹ to R⁵ represent the same radical, i.e. both radicals R¹ represent the same C₈-C₁₈ alkyl radical, both radicals R² and both radicals R³ represent hydrogen or the same C₁-C₄ alkyl or the same C₁-C₄ alkoxy radical, and both radicals R⁴ and both radicals R⁵ represent either hydrogen, methyl or methoxy. In addition, in the disulfonate compounds according to formula (I), both radicals X represent the same counterion, too, i.e. either both represent the same monovalent cation X⁺, or both X together are parts of the same polyvalent cation Xⁿ⁺, which, in such cases, is most preferably a bivalent cation X²⁺ such as Ca²⁺ or Mg²⁺. In these embodiments, the novel disulfonate compound according to formula (I) is mirror-symmetrical about an axis running through the central keto group (i.e. a vertical axis in the formula drawing).

In some particularly preferred embodiments it therefore applies that:
both radicals R¹ represent the same C₈-C₁₈ alkyl radical;
both radicals R² and both radicals R³ are each hydrogen or methoxy;
both radicals R⁴ and both radicals R⁵ are each hydrogen;
both radicals R⁶ are each hydrogen or methyl or are connected to one another to form an ethylene or propylene radical, thus forming a five-or six-membered ring comprising the carbonyl carbon; and
the radicals X each represent H⁺, Na⁺, NH₄⁺, or an organic ammonium ion which is most preferably (2-hydroxyethyl)trimethylammonium (choline) or triethanolammonium.

Most preferably, the sulfonate compound according to the first aspect of the present invention is selected from the following compounds:

1,5-bis(3-methoxy-4-octyloxyphenyl)-3-oxo-1,5-pentanedisulfonic acid diammonium salt (1)

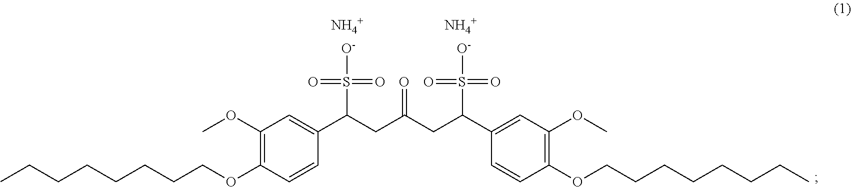 [SMILES: [NH4+] ; [NH4+] ; CCCCCCCCOc1ccc(C(CC(=O)CC(c2ccc(OCCCCCCCC)c(OC)c2)S(=O)(=O)[O-])S(=O)(=O)[O-])cc1OC]

1,5-bis(4-dodecyloxy-3-methoxyphenyl)-3-oxo-1,5-pentanedisulfonic acid diammonium salt (2)

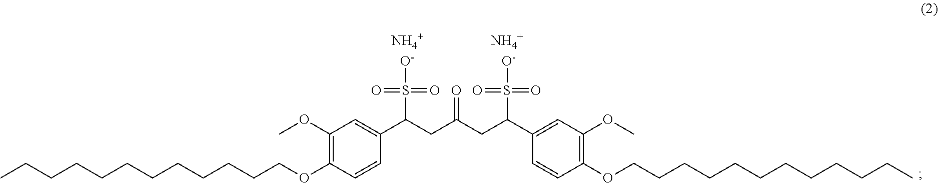 [SMILES: [NH4+] ; [NH4+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]

1,5-bis(3-methoxy-4-tetradecyloxyphenyl)-3-oxo-1,5-pentanedisulfonic acid diammonium salt (3)

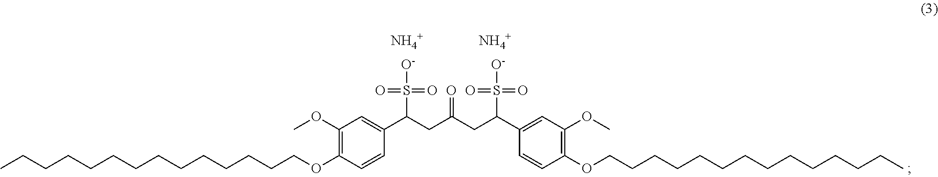 [SMILES: [NH4+] ; [NH4+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]

1,5-bis(4-hexadecyloxy-3-methoxyphenyl)-3-oxo-1,5-pentanedisulfonic acid diammonium salt (4)

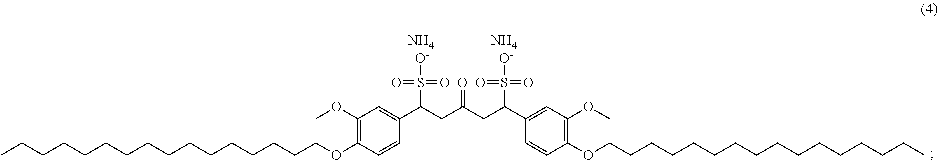 [SMILES: [NH4+] ; [NH4+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]

1,5-bis(3-methoxy-4-octadecyloxyphenyl)-3-oxo-1,5-pentanedisulfonic acid diammonium salt (5)

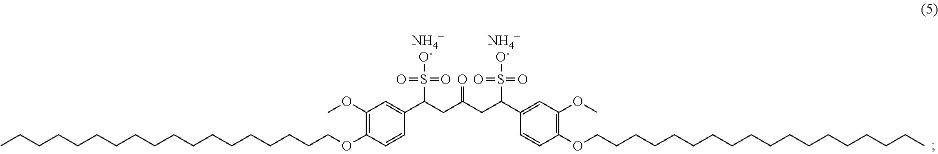 [SMILES: [NH4+] ; [NH4+] ; CCCCCCCCCCCCCCCCCCOc1ccc(C(CC(=O)CC(c2ccc(OCCCCCCCCCCCCCCCCCC)c(OC)c2)S(=O)(=O)[O-])S(=O)(=O)[O-])cc1OC]

1,5-bis(4-dodecyloxy-3-methoxyphenyl)-3-oxo-1,5-pentanedisulfonic acid disodium salt (6)

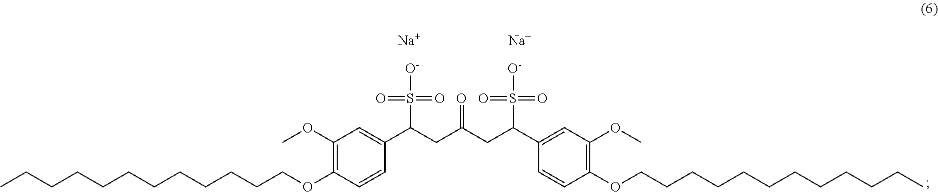 [SMILES: [Na+] ; [Na+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]

1,5-bis(3-methoxy-4-tetradecyloxyphenyl)-3-oxo-1,5-pentanedisulfonic acid disodium salt (7)

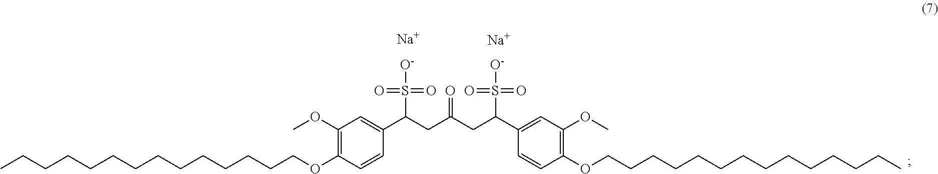 [SMILES: [Na+] ; [Na+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]

1,5-bis(4-hexadecyloxy-3-methoxyphenyl)-3-oxo-1,5-pentanedisulfonic acid disodium salt (8)

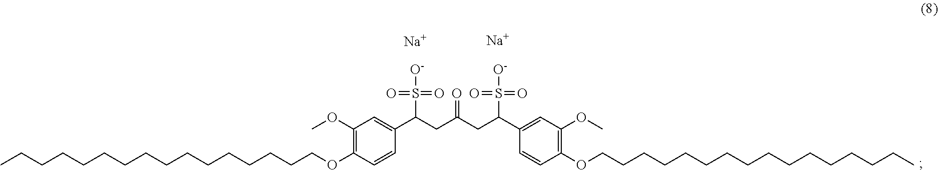 [SMILES: [Na+] ; [Na+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]

1,5-bis(3-methoxy-4-octadecyloxyphenyl)-3-oxo-1,5-pentanedisulfonic acid disodium salt (9)

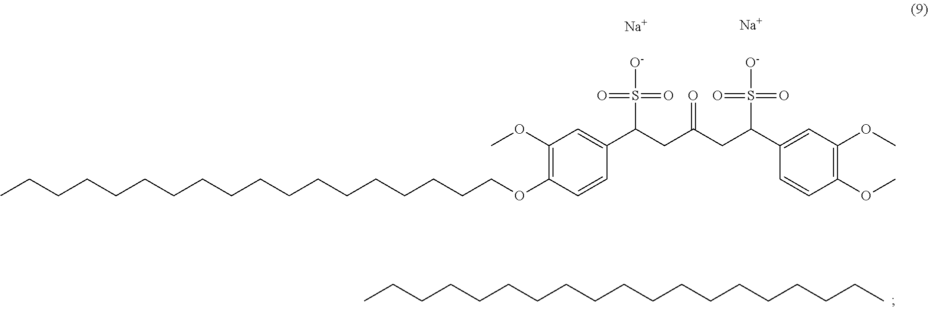

1,5-bis(4-hexadecyloxy-3-methoxyphenyl)-3-oxo-1,5-pentanedisulfonic acid dipotassium salt (10)

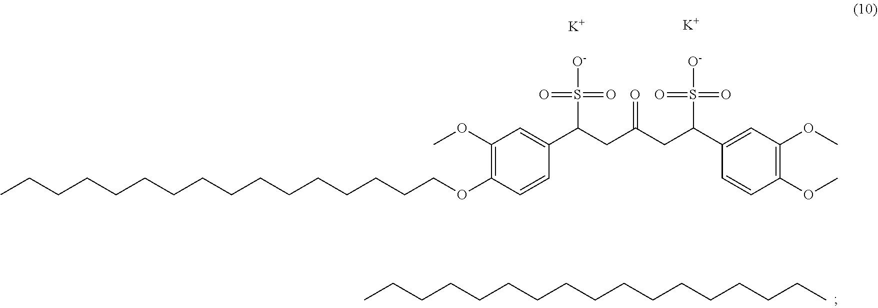

1,5-bis(4-dodecyloxy-3-methoxyphenyl)-3-oxo-1,5-pentanedisulfonic acid dicholine salt (11)

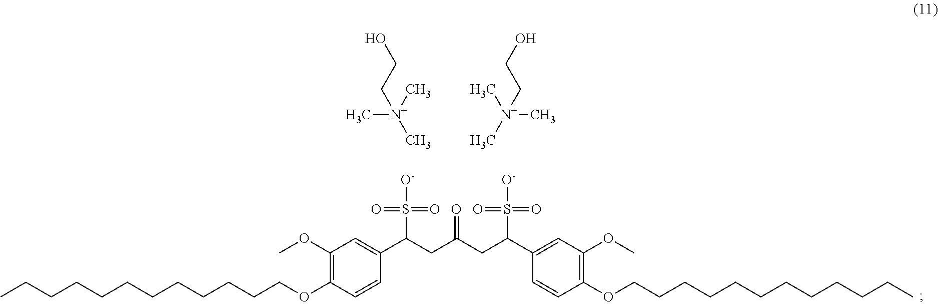

1,5-bis(4-dodecyloxy-3-methoxyphenyl)-3-oxo-1,5-pentanedisulfonic acid bis(triethanolammonium) salt (12)

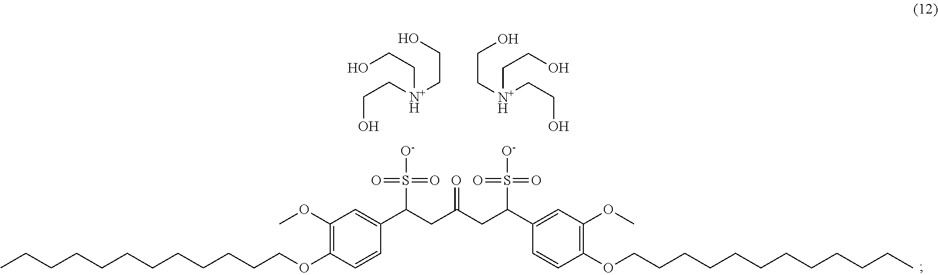

1,1′-(2-oxocyclopentane-1,3-diyl)-bis[(3-methoxy-4-octyloxyphenyl)methanesulfonic acid ammonium salt] (13)

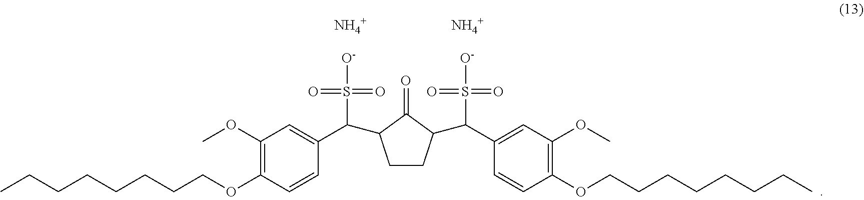 [SMILES: [NH4+] ; [NH4+] ; CCCCCCCCOc1ccc(C(C2CCC(C(c3ccc(OCCCCCCCC)c(OC)c3)S(=O)(=O)[O-])C2=O)S(=O)(=O)[O-])cc1OC]

In a second aspect, the present invention relates to a method for preparing a sulfonate compound according to the first aspect, which comprises the following steps:
1) reacting a 4-hydroxybenzaldehyde derivative according to the following formula (III):

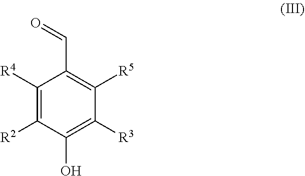

wherein
R² to R⁵ are each independently selected from hydrogen and linear, branched or cyclic hydrocarbon radicals having 1 to 26 carbon atoms, wherein optionally at least one carbon atom may be replaced by an oxygen or sulfur atom;
with a compound of the formula R¹—Y, wherein R¹ is selected from linear, branched or cyclic hydrocarbon radicals having 4 to 26 carbon atoms, wherein optionally at least one carbon atom may be replaced by an oxygen or sulfur atom, and Y represents a leaving group selected from halides and sulfonates, by means of an etherification reaction according to Williamson in the presence of a base in an organic solvent, resulting in a corresponding ether according to formula (IV):

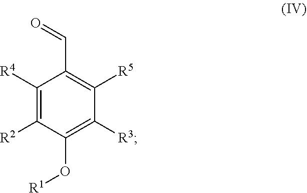

2) reacting the ether of formula (IV) with half an equivalent of acetone or an acetone derivative according to formula (V)

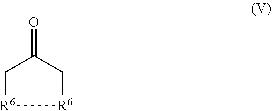

wherein each R⁶ is independently selected from hydrogen and saturated hydrocarbon radicals having 1 to 6 carbon atoms, wherein optionally the two radicals R⁶ may be connected, as indicated by the dashed line, to form a five-or six-membered ring comprising the carbonyl carbon atom, by means of a double, crossed aldol condensation reaction according to Claisen-Schmidt using an acidic or basic catalyst in an organic solvent, resulting in a corresponding unsaturated ketone according to formula (VI):

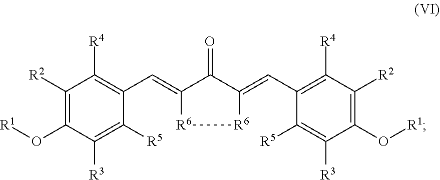

3) reacting the ketone of formula (VI) with a sulfonating agent in an alcoholic solvent for adding one or two equivalent(s) of hydrogen sulfite to the double bond(s) of the ketone of formula (VI), optionally followed by an ion exchange of the mono-or disulfonate thus obtained and introduction of predetermined counterions Xⁿ⁺, wherein n is ≥1, resulting in the sulfonate compound of formula (I) or (II).

In this way, according to the present invention, it is possible to synthesize the novel sulfonate compounds from hydroxybenzaldehyde derivatives according to formula (III) by a comparatively simple and inexpensive method that includes a sequence of known individual reactions. In preferred embodiments, the starting compounds are readily available products of lignin depolymerization, such as optionally substituted vanillin or syringaldehyde, and the method is carried out in the most environmentally friendly manner possible.

In some preferred embodiments of the method of the present invention, in step 1), the chloride or bromide, most preferably the bromide, is used as said leaving group Y; and/or K₂CO₃ is used as said base, most preferably two equivalents of K₂CO₃; and/or acetonitrile is used as said organic solvent, most preferably at reflux temperature.

As the leaving group Y, also a sulfonate, such as mesylate or tosylate, can be used; however, the use of long-chain fatty alcohol sulfonates would be uneconomical since these are already surfactants themselves, which is why the chloride or bromide, especially the bromide, is to be preferred. After a series of experiments with other polar aprotic solvents such as acetone, diethyl ether, and DMF, acetonitrile has proven to be a well suitable solvent, especially under reflux, as it resulted in the best conversion rates and simplified the subsequent purification of the product. For the latter reason, the hydroxybenzaldehyde derivative according to formula (III) is preferably used in a slight excess compared to the compound of the formula R¹—Y, for example in an excess of 10-15 mol %. In combination with the use of K₂CO₃ as a base, this offers the additional advantage that the KBr formed as a by-product can be collected and quasi recycled, i.e. can be used to synthesize other desired compounds of the formula R¹—Br. Since these are preferably fatty alkyl bromides, they can easily be produced from fatty alcohols using this KBr.

In some preferred embodiments of the method of the present invention, in step 2), lithium hydroxide monohydrate, LiOH.H₂O, is used as said basic catalyst, most preferably in an amount of 1-10 mol %; and/or a lower alcohol, an ether, or a mixture thereof, most preferably isopropanol, is used as said organic solvent, most preferably at a well-tested reaction temperature of 40-50° C.

As the basic catalyst other bases, such as NaOH or KOH, can also be used, or the reaction can be carried out using phase transfer catalysis, for example using tetra-n-butylammonium bromide (TBAB) as a catalyst. However, the inventors achieved the best results with regard to conversion and reaction time by using lithium hydroxide monohydrate, LiOH.H₂O. Similarily, instead of the preferred solvent, isopropanol, also ethers, other alcohols such as MeOH or EtOH, or mixtures of an alcohol with water or diethyl ether can be used. When using isopropanol, however, the reaction mixtures do not need to be heated to its reflux temperature (82.5° C.) in order to achieve high conversions in a short time.

In some preferred embodiments of the method of the present invention, in step 3),
a hydrogen sulfite or a disulfite, more preferably sodium disulfite Na₂S₂O₅, calcium hydrogen sulfite Ca(HSO₃)₂, ammonium hydrogen sulfite NH₄HSO₃ or trimethylammonium sulfite [(CH₃)₃N]₂SO₃, is used as said sulfonating agent; and/or
a mixture of a lower alcohol and water, more preferably aqueous methanol or isopropanol, is used as said alcoholic solvent; and/or
an amine, more preferably triethylamine, triethanolamine or choline hydroxide, is used as a catalyst.

In especially preferred embodiments, the hydrogen sulfite or disulfite is used in an amount of three equivalents of hydrogen sulfite and the amine catalyst is used in an amount of at least 20 mol %, each based on the ketone of formula (VI), and aqueous isopropanol is used as solvent under reflux, to obtain a disulfonate compound according to formula (I). For synthesizing a monosulfonate compound according to formula (II), the hydrogen sulfite or disulfite is only used in an amount of one equivalent of hydrogen sulfite in order to add only one sulfonic acid group to one of the two double bonds of the ketone according to formula (VI). In addition, by varying the hydrogen sulfite equivalents, it is also possible to produce mixtures of a disulfonate according to formula (I) and a monosulfonate according to formula (II), from which either the two products obtained can be isolated or the mixture as such, optionally after a subsequent ion exchange, can be used as a surfactant.

Unless the desired counterion has already been introduced into the sulfonate compound according to formula (I) or (II) in the course of the sulfonation, for example by using the corresponding amine as a catalyst, in step 3), the sulfonate adduct obtained is preferably first subjected to an ion exchange using an acidic ion exchange resin and water elution, for converting the sulfonate group(s) into the free sulfonic acid group(s), which optionally may be neutralized using an aqueous solution of the hydroxides of predetermined counterions Xⁿ⁺, to obtain the respective desired sulfonate compound according to formula (I) or (II).

In especially preferred embodiments of the method of the present invention, the steps 2) and 3) are carried out as a one-pot synthesis without isolating the ketone of formula (VI), by carrying out both reactions in the same lower alcohol as a solvent, wherein, after completion of the double, crossed aldol condensation reaction, for example by using 10 mol % LiOH.H₂O as a basic catalyst in an alcoholic solvent, especially isopropanol, for a reaction time of 12 h at 45° C., the sulfonating agent in the form of an aqueous solution, e.g. of NaHSO₃, the basic catalyst, e.g. an amine such as triethylamine or already the amine desired as a counterion, and optionally an additional amount of the same alcoholic solvent, are simply added, wherein the mixture is most preferably refluxed, e.g. for a reaction time of 12-14 h. The progress of the reaction can be monitored, for example, by the gradual disappearance of the coloring of the reaction mixture, e.g. an intense yellow color, which caused by the conjugated aromatic structure of the ketone of formula (VI).

In preferred embodiments, the reaction mixtures containing the inventive sulfonate compounds according to formula (I) or (II are worked up first by introducing O₂ at room temperature, in order to oxidize excess sulfite to sulfate, and subsequent addition of n-pentane and distilling off the ternary azeotrope of water, the lower alcohol, especially isopropanol, and pentane at an average temperature of e.g. 40-60° C., preferably using a Dean-Stark water separator, thereby removing the majority of water and lower alcohol. Subsequent drying on a rotary evaporator under vacuum produces a mostly slightly yellowish solid residue. Subsequently, this is preferably purified until discoloration by extraction with an organic solvent, such as acetone, CHCl₃, CH₂Cl₂ or Et₂O, for example using a Soxhlet extractor.

And in a third aspect, the present invention relates to the use of the novel sulfonate compounds according to formula (I) or (II), in which the total number of carbon atoms of the radicals R¹ to R⁵ should be at least 9, as surfactants.

### EXAMPLES

The present invention will be described in more detail below by way of examples, which, however, should not be construed as limiting the scope of protection. For illustration purposes, vanillin was used as a representative model compound for the common lignin depolymerization products preferred as starting substances in the method according to the invention and converted into the sulfonate compounds according to the invention. Further examples using its methoxy derivative, syringaldehyde, another typical aromatic aldehyde as a degradation product of lignin, are currently the subject of further experiments conducted by the inventors.

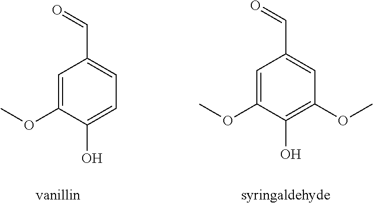

According to Reaction Scheme A below, vanillin (III) was first etherified with a series of different fatty alkyl halides R¹—Y, whereafter the ethers of the formula (IV) thus obtained were each subjected to a double, crossed aldol condensation reaction with half an equivalent of acetone or cyclopentanone (as indicated by the dashed bonds) to obtain doubly unsaturated ketones of the formula (VI), to which hydrogen sulfite was finally added in order to obtain the corresponding sulfonate compound according to the invention. In the last step, to date only two equivalents of hydrogen sulfite were added to both double bonds of the ketone in order to obtain disulfonates according to formula (I). However, it is obvious to the person skilled in the art that corresponding monosulfonates according to formula (II) or mixtures of both can also be obtained in an analogous manner, using only a smaller amount of sulfonating agent, as already mentioned before.

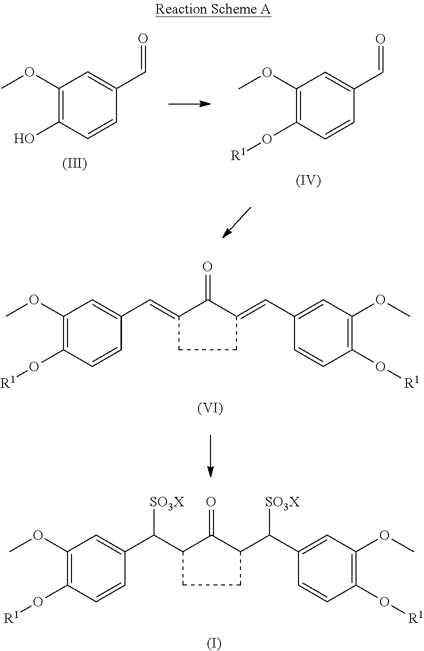

### Example 1

### Preparation of 1,5-bis(3-methoxy-4-octyloxyphenyl)-3-oxo-1,5-pentanedisulfonic Acid Diammonium Salt (1)

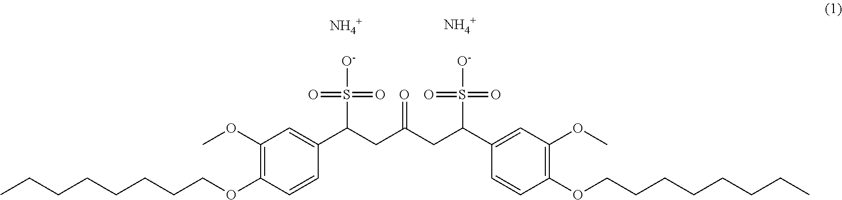 [SMILES: [NH4+] ; [NH4+] ; CCCCCCCCOc1ccc(C(CC(=O)CC(c2ccc(OCCCCCCCC)c(OC)c2)S(=O)(=O)[O-])S(=O)(=O)[O-])cc1OC]

### Step 1:

Vanillin (6.39 g, 42 mmol) was refluxed together with 1-bromooctane (6.76 g, 35 mmol) and oven-dried K₂CO₃ in 100 mL acetonitrile under nitrogen atmosphere for 48 h. The solvent was then removed on a rotary evaporator and the residue obtained was redissolved in 200 mL of a 1:1 mixture of diethyl ether (Et₂O) and petroleum ether (bp: 40-60° C.) and mixed with 50 mL of 0.5 M NaOH and then washed with 50 ml of water. The organic phase was dried over Na₂SO₄, whereafter the solvent was distilled off in vacuo and the residue was completely dried in a vacuum desiccator, giving the alkylated vanillin, 3-methoxy-4-octyloxybenzaldehyde, as a white powder (yield: 9.13 g; 98.7% of theory).

### Step 2:

4-Octyloxy-3-methoxybenzaldehyde (1.85 g, 7 mmol) was reacted in 14 mL isopropanol at 45° C. for 12 h with acetone (0.20 g, 3.5 mmol) in the presence of lithium hydroxide monohydrate, LiOH.H₂O, (14.7 g, 0.35 mmol) as a catalyst. The solid precipitate was then centrifuged off, washed 3 times with 6 mL MeOH and dried in vacuo, yielding the double adduct, 1,5-bis(3-methoxy-4-octyloxyphenyl)penta-1,4-dien-3-one, as a yellow powder (yield: 1.65 g; 85.4% of theory).

### Step 3:

### Variant 3.1

1,5-Bis(3-methoxy-4-octyloxyphenyl)penta-1,4-dien-3-one (1.1 g, 2 mmol) in 22 mL of isopropanol was reacted with 6 mL of a 1 M aqueous solution of sodium hydrogen sulfite, NaHSO₃, in the presence of triethylamine (0.04 g, 0.4 mmol) under reflux for 14 h. The solid precipitate was then centrifuged off and washed with MeOH. The combined organic phases were filtered through a 0.2 μm syringe filter, concentrated in vacuo and then passed through a bed of freshly activated acid ion exchange resin (DOWEX® 50WX8-100) and washed 3 times with 50 ml of water, whereafter concentrated NH₄OH solution was added in slight excess to produce the ammonium salt, which was filtered off and completely dried in a vacuum desiccator to give the title compound (1) as a white solid (yield: 1.34 g; 89.2% d. Th.).

### Variant 3.2

1,5-Bis(3-methoxy-4-octyloxyphenyl)penta-1,4-dien-3-one (28 mg, 0.05 mmol) was mixed with 0.1 mL of a 1.5 M aqueous solution of sodium hydrogen sulfite, NaHSO₃, and triethylamine (1 mg, 0.01 mmol) using a Teflon-coated stir bar in a microwave tube, which was tightly sealed and heated in a microwave reactor at 140° C. for 45 min. The reaction mixture was then dried in vacuo and the crude sample was dissolved in MeOD for analysis by NMR spectroscopy.

¹H NMR: δ_{H} (700 MHZ, MeOD) 6.97 (d, 1H, (C6, C12)), 6.89 (s, 1H, (C6^{m}, C12^{m})), 6.78 (m, 2H (C3, C4, C8, C9)), 6.70 (m, 2H, (C3^{m}, C4^{m}, C8^{m}, C9^{m})), 4.33 (m, 1H, (C13, C18)), 4.26 (m, 1H, (C13^{m}, C18^{m})), 3.96 (m, 4H, (C33, C41)), 3.81 (s, 3H, (C20, C22)), 3.76 (s, 3H, (C20^{m}, C22^{m})), 3.34-3.25 (m, 4H, (C14, C16)), 1.78 (m, 4H, (C34, C42)), 1.48 (m, 4H, (C35, C43)), 1.35 (m, 17H (C36, C37, C38, C39, C44, C45, C46, C47)), 0.92 (t, 6H (C40, C48)). ¹³C NMR: δ_{C} (176 MHz, MeOD) 207.4 (C15^{m}), 206.8 (C15), 150.3 (C1, C11), 150.1 (C1^{m}, C11^{m}), 149.4 (C2, C10), 149.3 (C2^{m}, C10^{m}), 130.5 (C5, C7), 130.3 (C5^{m}, C7^{m}), 123.0 (C4, C8), 122.5 (C4^{m}, C8^{m}), 114.8 (C6, C12), 114.1 (C3, C9), 113.9 (C3^{m}, C9^{m}), 70.2 (C33, C41), 70.1 (C33^{m}, C41^{m}), 62.6 (C13m, C18^{m}), 62.3 (C13, C18), 56.4 (C20, C22), 56.3 (C20^{m}, C22^{m}), 46.6 (C14^{m}, C16^{m}), 46.0 (C14, C16), 33.1 (C34^{m}, C42^{m}), 33.0 (C34, C42), 30.6 (C35^{m}, C43^{m}), 30.5 (C35, C43), 30.5 (C36, C44), 30.5 (C36^{m}, C44^{m}), 30.5 (C37^{m}, C45^{m}), 30.4 (C37, C45), 27.2 (C38^{m}, C46^{m}), 27.2 (C38, C46), 23.8 (C39^{m}, C47^{m}), 23.7 (C39, C47), 14.5 (C40, C48). Elemental Analysis: expected: C, 56.13; H, 8.07; N, 3.74; S, 8.56; found: C, 55.13; H, 8.33; N, 3.63; S, 8.36 HRMS: (ESI⁺, m/z) calculated for C₃₅H₆₁N₂O₁₁S₂ [M+H]⁺: 749.37168; found: 749.370948.

### Example 2

### Preparation of 1,5-bis(4-dodecyloxy-3-methoxyphenyl)-3-oxo-1,5-pentanedisulfonic Acid Diammonium Salt (2)

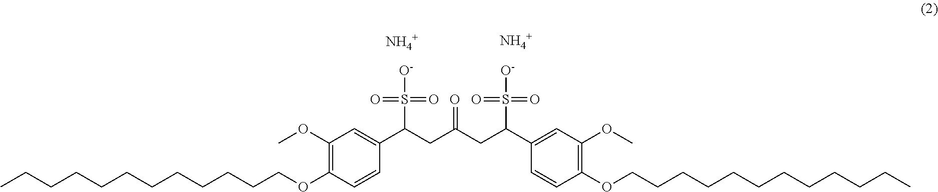 [SMILES: [NH4+] ; [NH4+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]

### Step 1:

The reaction was carried out in an analogous manner to that in Example 1, except that 1-bromododecane was used instead of 1-bromooctane, giving 4-dodecyloxy-3-methoxybenzaldehyde as a cream-colored powder (yield: 10.85 g; 96.8% of theory).

### Step 2:

The reaction was carried out in an analogous manner to that in Example 1, yielding 1,5-bis(4-dodecyloxy-3-methoxyphenyl)penta-1,4-dien-3-one as a yellow powder (yield: 20.0 g; 86.0% of theory).

### Step 3:

The reaction was carried out in an analogous manner to that in Example 1, except that only 1 mmol of 1,5-bis(4-dodecyloxy-3-methoxyphenyl)penta-1,4-dien-3-one was used, giving the title compound (2) as a cream-colored powder (yield: 0.75 g; 87.0% of theory).

1H NMR: δ_{H} (300 MHZ, MeOD) 6.94 (1 H, s, (C6, C12)), 6.85 (1 H, s, (C6^{m}, C12^{m})), 6.73 (2 H, d, J 1.1, (C3, C4, C8, C9)), 6.69-6.63 (2 H, m, (C3^{m}, C4^{m}, C8^{m}, C9^{m})), 4.34-4.27 (1 H, m, (C13, C18)), 4.26-4.17 (1 H, m, (C13^{m}, C18^{m})), 3.99-3.87 (4 H, m, (C14, C16)), 3.78 (2 H, s, (C20, C22)), 3.73 (4 H, s (C20^{m}, C22^{m})), 3.36-3.12 (4 H, m (C14, C16)), 1.84-1.67 (4 H, m (C34, C46)), 1.52-1.41 (4 H, m (C35, C47)), 1.41-1.24 (33 H, m), 0.89 (6 H, t, J6.8 (C44, C56)). ¹³C NMR: δ_{C} (75 MHZ, MeOD) 207.4 (C15^{m}), 206.8 (C15), 150.2 (C1, C11), 150.1 (C1^{m}, C11^{m}), 149.4 (C2, C10), 149.3 (C2^{m}, C10^{m}), 130.4 (C5, C7), 130.3 (C5^{m}, C7^{m}), 123.0 (C4, C8), 122.4 (C4^{m}, C8^{m})), 114.8 (C6, C12), 114.0 (C3, C9), 113.9 (C3^{m}, C9^{m}), 70.2 (C33, C45), 70.1 (C33^{m}, C45^{m}), 62.6 (C13^{m}, C18^{m}), 62.3 (C13, C18), 56.4 (C20, C22), 56.3 (C20^{m}, C22^{m}), 46.6 (C14^{m}, C16^{m}), 46.0 (C14, C16), 33.1 (C34, C42), 30.8 (C35, C36, C47, C48), 30.7 (C37, C38, C49, C50), 30.6 (C39, C51), 30.5 (C40, C52), 27.3 (C41, C53), 27.2 (C42, C54), 23.8 (C43, C55), 14.5 (C44, C56). Elemental Analysis: expected: C, 59.97; H, 8.90; N, 3.25; S, 7.45 found: C, 59.15; H, 8.92; N, 3.22; S, 7.13 HRMS: (ESI⁺, m/z) calculated for C₄₃H₇₇N₂O₁₁S₂ [M+H]⁺: 861.495738; found: 861.495738.

### Example 3

### Preparation of 1,5-bis(3-methoxy-4-tetradecyloxyphenyl)-3-oxo-1,5-pentanedisulfonic Acid Diammonium Salt (3)

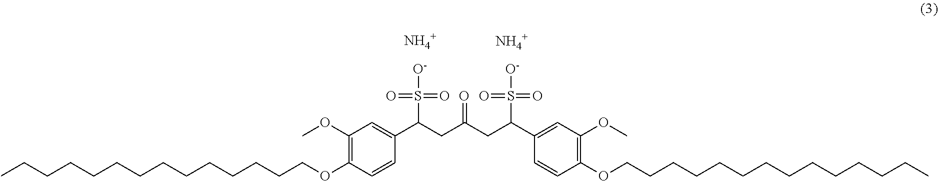 [SMILES: [NH4+] ; [NH4+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]

### Step 1:

The reaction was carried out in an analogous manner to that in Example 1, except that 1-bromotetradecane was used instead of 1-bromooctane, giving 3-methoxy-4-tetra-decyloxybenzaldehyde as a cream-colored powder (yield: 12.1 g; 99.2% of theory).

### Step 2:

The reaction was carried out in an analogous manner to that in Example 1, except that the crude product was recrystallized from boiling heptane after washing with MeOH, giving 1,5-bis(3-methoxy-4-tetradecyloxyphenyl)penta-1,4-dien-3-one as a yellow powder (yield: 1.17 g; 46.4% of theory).

### Step 3:

The reaction was carried out in an analogous manner to that in Example 2, giving the title compound (3) as a cream-colored powder (yield: 0.72 g; 78.3% of theory).

¹H NMR: δ_{H} (300 MHZ, MeOD) 6.97 (1 H, s (C6, C12)), 6.88 (1 H, s (C6^{m}, C12^{m})), 6.79-6.74 (2 H, m (C3, C4, C8, C9)), 6.74-6.62 (2 H, m (C3^{m}, C4^{m}, C8^{m}, C9^{m})), 4.39-4.29 (1 H, m (C13, C18)), 4.28-4.20 (1 H, m (C13^{m}, C18^{m})), 4.02-3.89 (4 H, m, (C33, C47)), 3.81 (3 H, s (C20, C22)), 3.76 (3 H, s (C20^{m}, C22^{m})), 3.36-3.16 (4 H, m (C14, C16)), 1.87-1.72 (4 H, m (C34, C48)), 1.55-1.46 (4 H, m (C35, C49)), 1.42-1.25 (42 H, m (C36-C45, C50-C59)), 0.92 (6 H, t, J6.9 (C46, C60)). ¹³C NMR: δ_{C} (75 MHZ, MeOD) 207.4 (C15^{m}), 206.9 (C15), 150.2 (C1, C11), 150.1 (C1^{m}, C11^{m}), 149.4 (C2, C10), 149.3 (C2^{m}, C10^{m})), 130.4 (C5, C7), 130.3 (C5^{m}, C7^{m}), 123.0 (C4, C8), 122.4 (C4^{m}, C8^{m}), 114.8 (C6, C12), 114.0 (C3, C9), 113.8 (C3^{m}, C9^{m})), 70.2 (C33, C47), 70.1 (C33^{m}, C47^{m}), 62.6 (C13^{m}, C18^{m}), 62.3 (C13, C18), 56.4 (C20, C22), 56.3 (C20^{m}, C22^{m}), 46.6 (C14^{m}, C16^{m}), 46.0 (C14, C16), 33.1 (C34, C48), 31.0-30.3 (m) (C35-C42, C49-C56), 27.3 (C43, C57), 27.2 (C44, C58), 23.8 (C45, C59), 14.5 (C46, C60). Elemental Analysis: expected: C, 61.54; H, 9.23; N, 3.05; S, 6.99 found: C, 59.58; H, 9.32; N, 2.93; S, 6.22 HRMS: (ESI⁻, m/z) calculated for C₄₇H₈₅N₂O₁₁S₂ [M+H]⁺: 917.558929; found: 917.557029.

### Example 4

### Preparation of 1,5-bis(4-hexadecyloxy-3-methoxyphenyl)-3-oxo-1,5-pentane-disulfonic Acid Diammonium Salt (4)

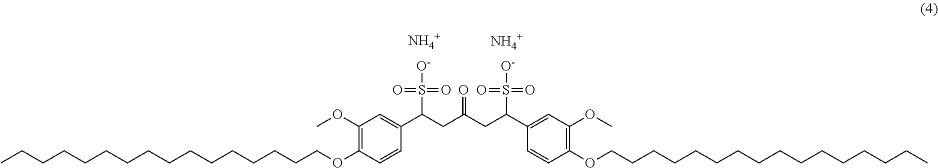 [SMILES: [NH4+] ; [NH4+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]

### Step 1:

The reaction was carried out in an analogous manner to that in Example 1, except that 1-bromohexadecane was used instead of 1-bromooctane, giving 4-hexadecyloxy-3-methoxybenzaldehyde as a cream-colored powder (yield: 13.0 g; 98.7% of theory).

### Steps 2 & 3 (One-Pot Synthesis):

4-Hexadecyloxy-3-methoxybenzaldehyde (2.63 g, 7 mmol) was reacted in 14 mL isopropanol with acetone (0.20 g, 3.5 mmol) in the presence of lithium hydroxide monohydrate, LiOH. H₂O, (14.7 g, 0.35 mmol) as a catalyst at 45° C. for 12 h. Triethylamine (0.07 g, 0.7 mmol), 10.5 mL of a 1 M aqueous solution of ammonium hydrogen sulfite, NH₄HSO₃, and further 38.5 mL of isopropanol were then added, whereafter the reaction mixture was refluxed for 14 h with vigorous stirring. The reaction mixture was then placed under an O₂ atmosphere using a balloon filled with oxygen gas in order to oxidize unreacted sulfite to sulfate. 50 mL of pentane were then added and the water was distilled off as an azeotrope using a Dean-Stark apparatus at 50° C., whereafter the liquid residue was concentrated in vacuo on a rotary evaporator. Acetone was added to the resulting residue and extracted using a Soxhlet extractor. Inorganic salts were removed by washing with MeOH, whereafter the solvent was removed from the remaining residue on a rotary evaporator and the residue was completely dried in a vacuum desiccator, giving the title compound (4) as a cream-colored powder (yield: 2.17 g; 63.8% of theory).

1H NMR: δ_{H} (300 MHZ, MeOD) 6.94 (s, 1H, C6, C12), 6.85 (d, J=1.7 Hz, 1H, (C6m, C12^{m})), 6.73 (d, J=1.1 Hz, 1H, (C3, C4, C8, C9)), 6.65 (d, J=2.3 Hz, 3H, (C3^{m}, C4^{m}, C8^{m}, C9^{m})), 4.31 (dd, J=9.3, 5.2 Hz, 1H, (C13, C18)), 4.22 (dd, J=10.4, 4.0 Hz, 1H, (C13^{m}, C18^{m})), 3.94 (t, J=6.6, 6.6 Hz, 4H (C33, C49)), 3.78 (s, 2H (C20, C22), 3.74 (s, 4H (C20^{m}, C22^{m})), 1.76 (s, 4H (C34, C50)), 1.29 (s, 44H (C35-C47, C51-C63), 0.89 (t, J=6.8 Hz, 7H (C48, C64) Elemental Analysis: expected: C, 62.93; H, 9.53; N, 2.88; S, 6.59 found: C, 58.93; H, 9.50; N, 2.77; S, 6.19.

### Example 5

### Preparation of 1,5-bis(3-methoxy-4-octadecyloxyphenyl)-3-oxo-1,5-pentanedisulfonic Acid Diammonium Salt (5)

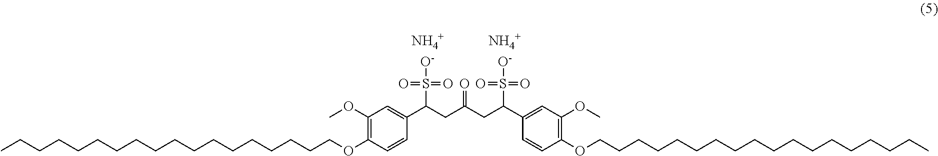 [SMILES: [NH4+] ; [NH4+] ; CCCCCCCCCCCCCCCCCCOc1ccc(C(CC(=O)CC(c2ccc(OCCCCCCCCCCCCCCCCCC)c(OC)c2)S(=O)(=O)[O-])S(=O)(=O)[O-])cc1OC]

### Step 1:

The reaction was carried out in an analogous manner to that in Example 1, except that 1-bromooctadecane was used instead of 1-bromooctane and 10 times the solution volume was used, giving 3-methoxy-4-octadecyloxybenzaldehyde as a cream-colored powder (yield: 1.38 g; 97.6% of theory).

### Steps 2 & 3 (One-Pot Synthesis):

The reaction was carried out in an analogous manner to that in Example 4 using 3-methoxy-4-octadecyloxybenzaldehyde instead of 4-hexadecyloxy-3-methoxybenzaldehyde, giving the title compound (5) as a cream-colored powder (yield: 11.9 g; 33.0% of theory).

¹H NMR (300 MHZ, MeOD) δ 6.94 (s, 1H), 6.85 (d, J=1.6 Hz, 1H), 6.75-6.70 (m, 2H), 6.65 (d, J=2.5 Hz, 2H), 4.32 (dd, J=9.3, 5.2 Hz, 1H), 4.22 (dd, J=10.3, 4.1 Hz, 1H), 3.94 (t, J=6.6, 6.6 Hz, 4H), 3.79 (s, 3H), 3.74 (s, 3H), 1.82-1.72 (m, 5H), 1.54-1.22 (m, 62H), 0.88 (t, J=7.0 Hz, 6H).

### Example 6

### Preparation of 1,5-bis(4-dodecyloxy-3-methoxyphenyl)-3-oxo-1,5-pentanedisulfonic Acid Disodium Salt (6)

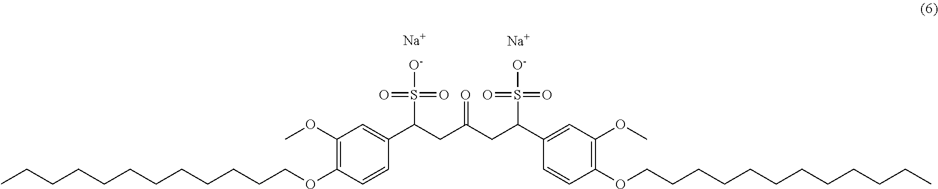 [SMILES: [Na+] ; [Na+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]

### Step 1:

The synthesis and the product were identical to Example 2.

### Steps 2 & 3 (One-Pot Synthesis):

The reaction was carried out in an analogous manner to that in Example 4, except that 4-dodecyloxy-3-methoxybenzaldehyde was used instead of 4-hexadecyloxy-3-methoxybenzaldehyde and sodium hydrogen sulfite, NaHSO3, was used as a sulfonating agent instead of ammonium hydrogen sulfite, NH₄HSO₃, giving the title compound (6) as a cream-colored powder (yield: 1.70 g; 55.7% of theory).

¹H NMR (300 MHZ, MeOD) 6.95 (s, 1H), 6.85 (s, 1H), 6.77-6.71 (m, 2H), 6.65 (d, J=2.0 Hz, 2H), 4.31 (dd, J=8.6, 5.8 Hz, 1H), 4.22 (dd, J=10.3, 4.1 Hz, 1H), 3.99-3.85 (m, 4H), 3.79 (s, 3H), 3.74 (s, 3H), 1.84-1.69 (m, 4H), 1.55-1.41 (m, 4H), 1.41-1.21 (m, 42H), 0.88 (t, J=7.0 Hz, 6H).

### Example 7

### Preparation of 1,5-bis(3-methoxy-4-tetradecyloxyphenyl)-3-oxo-1,5-pentanedisulfonic Acid Disodium Salt (7)

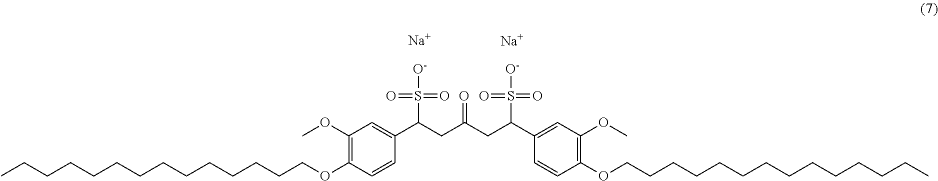 [SMILES: [Na+] ; [Na+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]

### Step 1:

The synthesis and the product were identical to Example 3.

### Steps 2 & 3 (One-Pot Synthesis);

The reaction was carried out analogously to that in Example 4, except that 3-methoxy-4-tetradecyloxybenzaldehyde was used instead of 4-hexadecyloxy-3-methoxybenzaldehyde and sodium hydrogen sulfite, NaHSO₃, was used as a sulfonating agent instead of ammonium hydrogen sulfite, NH₄HSO₃, giving the title compound (6) as a cream-colored powder (yield: 1.70 g; 55.7% of theory).

¹H NMR (300 MHz, MeOD) δ 6.95 (s, 1H), 6.86 (d, J=1.5 Hz, 2H), 6.74 (s, 2H), 6.66 (d, J=1.8 Hz, 3H), 4.32 (dd, J=9.6, 4.8 Hz, 1H), 4.24 (dd, J=10.4, 3.9 Hz, 2H), 3.94 (d, J=7.0 Hz, 4H), 3.79 (s, 3H), 3.74 (s, 4H), 3.39-3.33 (m, 1H), 3.28-3.14 (m, 2H), 1.84-1.69 (m, 4H), 1.56-1.42 (m, 4H), 1.41-1.20 (m, 44H), 0.91 (t, J=6.3, 6.3 Hz, 6H). HRMS: (ESI⁻, m/z) calculated for C₄₇H₇₇N₂O₁₁S₂ [M+H]⁺: 927.469721; found: 927.469727.

### Example 8

### Preparation of 1,5-bis(4-hexadecyloxy-3-methoxyphenyl)-3-oxo-1,5-pentane-disulfonic Acid Disodium Salt (8)

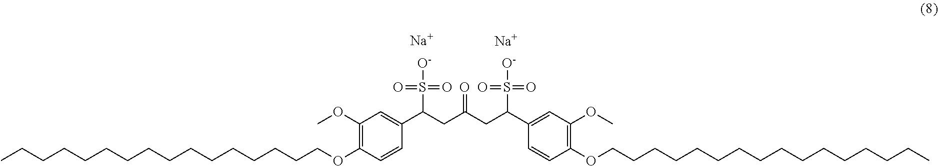 [SMILES: [Na+] ; [Na+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]

### Step 1:

The synthesis and the product were identical to Example 4.

### Steps 2 & 3 (One-Pot Synthesis):

The reaction was carried out in an analogous manner to that in Example 4, except that sodium hydrogen sulfite, NaHSO₃, was used as the sulfonating agent instead of ammonium hydrogen sulfite, NH₄HSO₃, giving the title compound (8) as a cream-colored powder (yield: 2.66 g; 77.3% of theory).

¹H NMR: δ_{H} (700 MHZ, MeOD) δ 6.96 (s, 1H), 6.88 (d, J=1.8 Hz, 1H), 6.75 (d, J=1.2 Hz, 1H), 6.70-6.65 (m, 3H), 4.34 (dd, J=10.3, 4.2 Hz, 1H), 4.25 (dd, J=10.8, 3.7 Hz, 1H), 3.96 (t, J=6.5 Hz, 4H), 3.80 (s, 2H), 3.76 (s, 4H), 3.36-3.33 (m, 1H), 3.31-3.20 (m, 2H), 1.78 (dt, J=8.7, 6.7 Hz, 4H), 1.49 (td, J=8.0, 7.6, 4.2 Hz, 4H), 1.42-1.36 (m, 5H), 1.35-1.28 (m, 49H), 0.91 (t, J=7.0 Hz, 5H). 13C NMR: δ_{C} (75 MHz, MeOD) (176 MHZ, MeOD) δ 207.5, 207.0, 150.2, 150.1, 149.4, 149.3, 130.4, 130.3, 123.1, 122.4, 114.8, 114.0, 113.9, 70.2, 70.1, 62.6, 62.3, 46.6, 46.0, 33.1 (d, J=2.1 Hz), 30.9, 30.8, 30.8, 30.8, 30.8, 30.7, 30.6, 30.6, 30.5-30.4 (m), 27.3, 27.2, 23.8, 14.5. Elemental Analysis: expected: C, 62.30; H, 8.61; Na, 4.68; S, 6.52 found: C, 60.68; H, 9.07; S, 5.49 HRMS: (ESI⁺, m/z) calculated for C₅₁H₈₅Na₂O₁₁S₂ [M+H]⁺: 983.532321 found: 983.531847.

### Example 9

### Preparation of 1,5-bis(3-methoxy-4-octadecyloxyphenyl)-3-oxo-1,5-pentanedisulfonic Acid Disodium Salt (9)

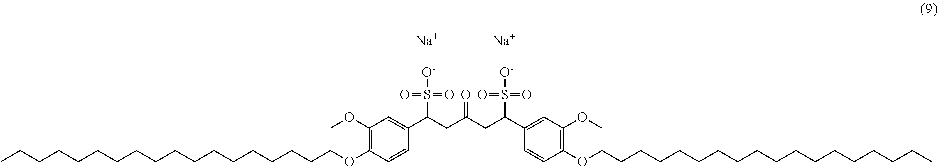 [SMILES: [Na+] ; [Na+] ; CCCCCCCCCCCCCCCCCCOc1ccc(C(CC(=O)CC(c2ccc(OCCCCCCCCCCCCCCCCCC)c(OC)c2)S(=O)(=O)[O-])S(=O)(=O)[O-])cc1OC]

### Step 1:

The synthesis and the product were identical to Example 5.

### Steps 2 & 3 (One-Pot Synthesis):

The reaction was carried out in an analogous manner to that in Example 8, except that 3-methoxy-4-octadecyloxybenzaldehyde was used instead of 4-hexadecyloxy-3-methoxybenzaldehyde, giving the title compound (9) as a cream-colored powder (yield: 1.05 g; 28.9% of theory).

¹H NMR: δ_{H} (300 MHZ, MeOD) δ 6.96 (s, 1H), 6.87 (d, J=1.6 Hz, 1H), 6.78-6.71 (m, 2H), 6.70-6.61 (m, 2H), 4.40-4.26 (m, 1H), 3.96 (dd, J=7.6, 5.5 Hz, 4H), 3.80 (s, 2H), 3.76 (s, 3H), 1.84-1.70 (m, 5H), 1.30 (d, J=3.2 Hz, 56H), 0.96-0.87 (m, 6H). Elemental Analysis: expected: C, 63.55; H, 8.92; S, 6.17 found: C, 59.72; H, 9.06; S, 6.78 HRMS: (ESI⁺, m/z) calculated for C₅₅H₉₃Na₂O₁₁S₂ [M+H]⁺1039.594921 found: 1039.595834.

### Example 10

### Preparation of 1,5-bis(4-hexadecyloxy-3-methoxyphenyl)-3-oxo-1,5-pentanedisulfonic Acid Dipotassium Salt (10)

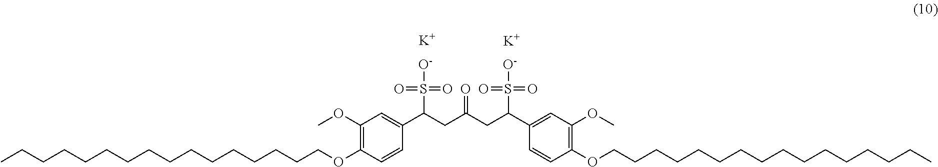 [SMILES: [K+] ; [K+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]

### Step 1:

The synthesis and the product were identical to Example 4.

### Steps 2 & 3 (One-Pot Synthesis):

The reaction was carried out in an analogous manner to that in Example 4, except that potassium hydrogen sulfite, KHSO₃, was used as a sulfonating agent instead of ammonium hydrogen sulfite, NH₄HSO₃, giving the title compound (10) as a cream-colored powder (yield: 1.16 g; 32.8% of theory).

¹H NMR: δ_{H} (300 MHZ, MeOD) 6.94 (s, 1H, C6, C12), 6.85 (d, J=1.7 Hz, 1H, (C6^{m}, C12^{m})), 6.73 (d, J=1.1 Hz, 1H, (C3, C4, C8, C9)), 6.65 (d, J=2.3 Hz, 3H, (C3^{m}, C4^{m}, C8^{m}, C9^{m})), 4.31 (dd, J=9.3, 5.2 Hz, 1H, (C13, C18)), 4.22 (dd, J=10.4, 4.0 Hz, 1H, (C13^{m}, C18^{m})), 3.94 (t, J=6.6, 6.6 Hz, 4H (C33, C49)), 3.78 (s, 2H (C20, C22), 3.74 (s, 4H (C20^{m}, C22^{m})), 1.76 (s, 4H (C34, C50)), 1.29 (s, 44H (C35-C47, C51-C63), 0.89 (t, J=6.8 Hz, 7H (C48, C64) Elemental Analysis: expected: C, 60.32; H, 8.34; K, 7.70; S, 6.31 found: C, 56.57; H, 8.62; S, 8.00 HRMS: (ESI⁺, m/z) calculated for C₅₁H₈₅K₂O₁₁S₂ [M+H]⁺: 1015.480195 found: 1015.480682.

### Example 11

### Preparation of 1,5-bis(4-dodecyloxy-3-methoxyphenyl)-3-oxo-1,5-pentanedisulfonic Acid Dicholine Salt (11)

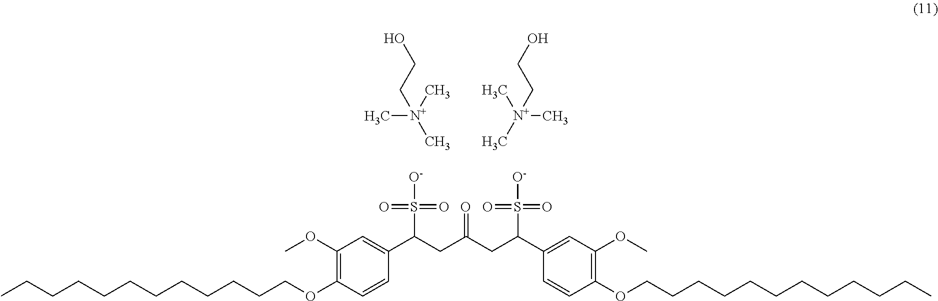

### Step 1:

The synthesis and the product were identical to Example 2.

### Step 2:

The synthesis and the product were identical to Example 2.

### Step 3:

The reaction was carried out in an analogous manner to that in Example 1, Variant 3.1, except that only 0.5 mmol of 1,5-bis(4-dodecyloxy-3-methoxyphenyl)penta-1,4-dien-3-one were used and that an aqueous solution of choline hydroxide was added in slight excess instead of the NH₄OH solution after passing the ion exchange resin, giving the title compound (11) as a cream-colored powder (yield: 0.52 g; 100.7% of theory).

¹H NMR: δ_{H} (300 MHZ, MeOD) 6.95 (d, J=1.2 Hz, 1H), 6.86 (d, J=1.6 Hz, 1H), 6.75 (d, J=1.1 Hz, 2H), 6.66 (d, J=2.4 Hz, 2H), 4.31 (dd, J=9.2, 5.1 Hz, 1H), 4.22 (dd, J=10.3, 4.1 Hz, 1H), 4.01-3.89 (m, 8H), 3.79 (s, 3H), 3.74 (s, 3H), 3.48-3.39 (m, 4H), 3.16 (s, 20H), 1.82-1.69 (m, 4H), 1.29 (q, J=5.2, 5.2, 4.2 Hz, 35H), 0.89 (d, J=6.9 Hz, 6H). ¹³C NMR: δ_{C} (75 MHZ, MeOD) 207.4, 206.9, 150.2, 150.1, 149.4, 149.3, 130.6, 130.4, 123.1, 122.5, 114.8, 114.1, 113.9, 70.2, 70.1, 69.1-68.9 (m), 62.3, 57.1, 56.5, 56.4, 54.8-54.5 (m), 46.6, 46.0, 33.1, 31.0-30.4 (m), 27.3, 27.2, 23.8, 14.5. Elemental Analysis: expected: C, 61.48; H, 9.54; N, 2.71; S, 6.19 found: C, 58.31; H, 9.56; N, 3.34; S, 6.02.

### Example 12

### Preparation of 1,5-bis(4-dodecyloxy-3-methoxyphenyl)-3-oxo-1,5-pentanedisulfonic Acid Bis(triethanolammonium) Salt (12)

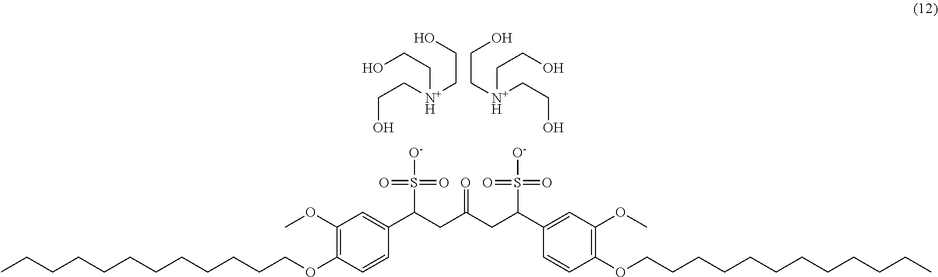

### Step 1:

The synthesis and the product were identical to Example 2.

### Step 2:

The synthesis and the product were identical to Example 2.

### Step 3:

The reaction was carried out in an analogous manner to that in Example 1, Variant 3.1, except that only 0.5 mmol of 1,5-bis(4-dodecyloxy-3-methoxyphenyl)penta-1,4-dien-3one were used and that an aqueous solution of triethanolamine was added in slight excess instead of the NH₄OH solution after the passage of the ion exchange resin, giving the title compound (12) as a cream-colored powder (yield: 0.55 g; 98.5% of theory).

1H NMR: δ_{H} (300 MHZ, MeOD) 7.0 (s, 1H), 6.9 (d, J=1.6 Hz, 1H), 6.7 (d, J=1.1 Hz, 2H), 6.7 (d, J=2.1 Hz, 2H), 4.4-4.3 (m, 1H), 4.3-4.2 (m, 1H), 3.9 (t, J=6.6, 6.6, 1.6 Hz, 4H), 3.9-3.8 (m, 14H), 3.8 (s, 3H), 3.7 (s, 3H), 3.4 (q, J=5.2, 5.2, 5.0 Hz, 12H), 1.8-1.7 (m, 4H), 1.5-1.4 (m, 4H), 1.4-1.2 (m, 34H), 0.9-0.9 (m, 6H). ¹³C NMR: δ_{C} (75 MHz, MeOD) 207.4, 206.9, 150.2, 150.1, 149.3, 130.5, 130.3, 123.1, 122.4, 114.8, 114.0, 113.8, 70.2, 70.1, 62.6, 56.9, 56.7, 56.4, 56.3, 46.6, 33.1, 30.8, 30.8, 30.7, 30.5, 27.3, 27.2, 23.8, 14.5. Elemental Analysis: expected: C, 58.80; H, 8.79; N, 2.49; S, 5.71 found: C, 57.01; H, 9.21; N, 2.71; S, 5.29.

### Example 13

### Preparation of 1,1′-(2-oxocyclopentane-1,3-diyl)-bis[(3-methoxy-4-octyloxyphenyl)methanesulfonic Acid Ammonium Salt] (13)

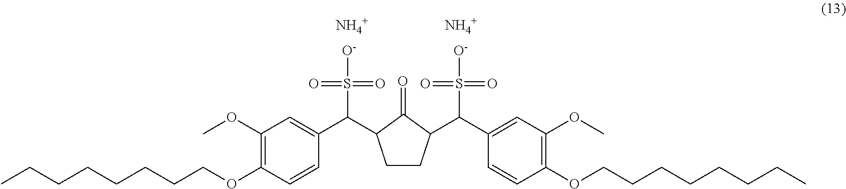 [SMILES: [NH4+] ; [NH4+] ; CCCCCCCCOc1ccc(C(C2CCC(C(c3ccc(OCCCCCCCC)c(OC)c3)S(=O)(=O)[O-])C2=O)S(=O)(=O)[O-])cc1OC]

### Step 1:

The synthesis and the product were identical to Example 1.

### Step 2:

The reaction was carried out in an analogous manner to that in Example 1, except that the 4-octyloxy-3-methoxybenzaldehyde was reacted with cyclopentanone (0.29 g, 3.5 mmol) instead of acetone and the crude product precipitate was washed with hexane instead of MeOH after centrifuging, giving 2,5-bis(3-methoxy-4-octyloxybenzylidene)cyclopentan-1-one as a yellow powder (yield: 1 78 g; 87.9% of theory).

### Step 3:

The reaction was carried out in an analogous manner to that in Example 1, Variant 3.1, except that 2,5-bis(3-methoxy-4-octyloxybenzylidene)cyclopentan-1-one (0.29 g, 0.5 mmol) was reacted with 6.2 mL of a 0.8 M aqueous solution of sulfurous acid, H₂SO₃, in the presence of triethylamine (0.59 g, 10 mmol), giving the title compound (13) in the form of yellowish crystals (yield: 0.37 g; 96.1% of theory).

1H NMR: δ_{H} (300 MHZ, MeOD) 7.19-7.03 (m, 2H), 6.98-6.74 (m, 3H), 6.48-6.32 (m, 1H), 4.63-4.37 (m, 2H), 4.07-3.84 (m, 5H), 3.83-3.67 (m, 6H), 3.39-3.31 (m, 2H), 3.23 (d, J=7.9 Hz, 1H), 2.61 (d, J=30.9 Hz, 1H), 2.53 (s, 1H), 2.35 (q, J=11.3, 9.5 Hz, 1H), 1.91 (ddd, J=11.7, 8.2, 3.7 Hz, 1H), 1.86-1.70 (m, 4H), 1.60-1.39 (m, 6H), 1.37-1.23 (m, 16H), 0.94-0.86 (m, 6H). ¹³C NMR: δ_{C} (75 MHZ, MeOD) 215.8, 148.8, 148.6, 147.9, 130.5, 127.3, 126.5, 122.9, 122.0, 114.4, 113.6, 113.3, 112.5, 111.8, 68.8, 68.7, 68.4, 64.7, 64.3, 63.9, 55.0, 54.8, 53.4, 51.4, 49.6, 47.3, 47.0, 46.7, 31.7, 31.6, 31.6, 29.3, 29.1, 29.0, 29.0, 29.0, 28.9, 25.9, 25.7, 23.3, 22.4, 22.3, 13.0. Elemental Analysis: expected: C, 57.34; H, 8.06; N, 3.61; S, 8.27; found: C, 57.34; H, 8.50; N, 4.50; S, 6.85 HRMS: (ESI⁺, m/z) calculated for C₃₇H₆₃N₂O₁₁S₂ [M+H]⁺: 775.386779; found: 775.385622.

An exact assignation of the peaks in the NMR spectra was not possible since the compound comprises four stereocenters and the product represents a mixture of diastereomers.

### Example 14

### Tests Regarding the Suitability of the Isolated Sulfonate Compounds According to Formulae (I) and (II) as Surfactants

As usual, the critical micelle concentration (CMC), i.e. the concentration at which micelles can form, was determined as a parameter for the surfactant properties of the novel sulfonate compounds using a K100C Force Tensiometer from Krüss Scientific using the Wilhelmy plate method at 25° C. and natural pH. For comparison, dodecyldimethylamine sulfonate (“Comparison”) was measured under the same conditions. The results are given in Table 1 below, with lower values indicating a stronger surfactant effect of the respective substance.

TABLE 1

| Compound | CMC (mg/L) |
|---|---|
| (1) | 3.2 × 10² |
| (2) | 64 |
| (3) | 34 |
| V1 | 6.8 × 10² |

It can be seen that the three tested examples according to the invention show lower—and in the majority even significantly lower—CMC values than the comparison substance, which is used in a large number of commercially available products. However, the CMC value of 0.32 g/l for the disulfonate (1) from Example 1, which contains the lowest number of carbon atoms in the radicals R¹ to R⁵ among the three compounds according to the invention due to the octyl radical R¹, is still less than half of the commercially available surfactant of the comparative example.

Due to the analogies or high similarities in the substitution patterns of the other compounds according to the invention that have not yet been tested, a person skilled in this art may expect that a strong surfactant effect will also be consistently detectable for these compounds. This applies equally to the substances (1) to (13) synthesized herein based on vanillin as well as to any other compounds that can be produced according to the present invention using similar lignin degradation products as starting materials, especially since the majority of them even have a larger number of carbon atoms in the radicals R¹ to R⁵, such as syringaldehyde, which, compared to vanillin, comprises an additional methoxy substituent as the radical R³.

The present invention thus provides a group of novel sulfonate compounds which are obtainable by relatively simple synthetic steps in an economical and environmentally friendly manner and the vast majority of which are suitable for use as surfactants.

1. A sulfonate compound according to formula (I) or (II): 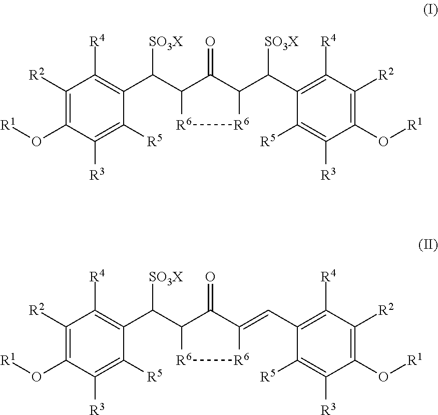
wherein
each R¹ is selected from linear, branched or cyclic hydrocarbon radicals having 4 to 26 carbon atoms, wherein optionally at least one carbon atom may be replaced by an oxygen or sulfur atom;
R² to R⁵ are each independently selected from hydrogen and linear, branched or cyclic hydrocarbon radicals having 1 to 26 carbon atoms, wherein optionally at least one carbon atom may be replaced by an oxygen or sulfur atom;
each R⁶ is independently selected from hydrogen and saturated hydrocarbon radicals having 1 to 6 carbon atoms, wherein optionally the two radicals R⁶ may be connected, as indicated by the dashed line, to form a five-or six-membered ring comprising the carbonyl carbon atom; and
each X is independently selected from mono-or polyvalent cations Xⁿ⁺, wherein n is ≥1, including H⁺, wherein, in formula (I), optionally both X together may represent a polyvalent cation.

2. The sulfonate compound according to claim 1, wherein
each R¹ represents C₆-C₂₂ alkyl; and/or
R² and R³ are each independently selected from hydrogen, C₁-C₂₂ alkyl and C₁-C₂₂ alkoxy; and/or
R⁴ and R⁵ are selected from hydrogen, methyl and methoxy; and/or
each R⁶ is selected from hydrogen, methyl and ethyl; and/or
each X represents H⁺, Na⁺, K⁺, NH₄⁺or an organic ammonium ion.

3. The sulfonate compound according to claim 1, wherein
R¹ represents C₈-C₁₈ alkyl; and/or
R² and R³ are each independently selected from hydrogen, C₁-C₄ alkyl and C₁-C₄ alkoxy;
R⁴ and R⁵ each are hydrogen.

4. The sulfonate compound according to claim 1, wherein
both R¹ represent the same C₈-C₁₈ alkyl radical;
one of R² and R³ is hydrogen and the other is methoxy or both of them are methoxy;
R⁴ and R⁵ each are hydrogen;
both radicals R⁶ are hydrogen or methyl, or they are connected to form an ethylene or propylene radical, thus forming a five-or six-membered ring comprising the carbonyl carbon atom; and
each X represents H⁺, Na⁺, NH₄⁺ or an organic ammonium ion, which optionally may be (2-hydroxyethyl)trimethylammonium (choline) or triethanolammonium.

5. The sulfonate compound according to claim 1, wherein it is selected from the following compounds:
1,5-bis(3-methoxy-4-octyloxyphenyl)-3-oxo-1,5-pentanedisulfonic acid diammonium salt (1) 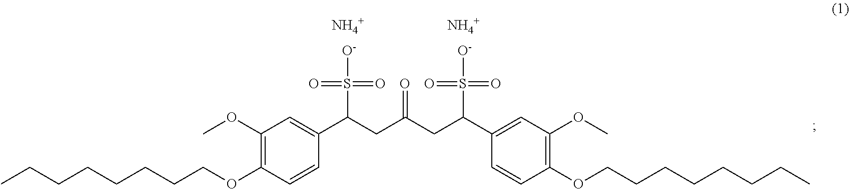 [SMILES: [NH4+] ; [NH4+] ; CCCCCCCCOc1ccc(C(CC(=O)CC(c2ccc(OCCCCCCCC)c(OC)c2)S(=O)(=O)[O-])S(=O)(=O)[O-])cc1OC]
1,5-bis(4-dodecyloxy-3-methoxyphenyl)-3-oxo-1,5-pentanedisulfonic acid diammonium salt (2) 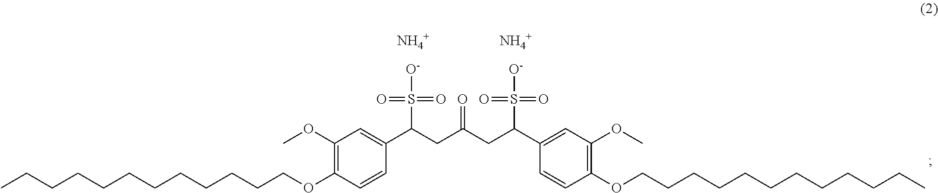 [SMILES: [NH4+] ; [NH4+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]
1,5-bis(3-methoxy-4-tetradecyloxyphenyl)-3-oxo-1,5-pentanedisulfonic acid diammonium salt (3) 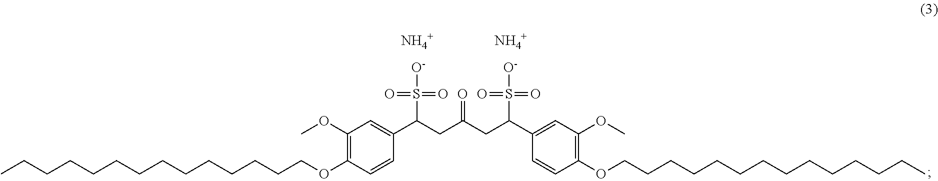 [SMILES: [NH4+] ; [NH4+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]
1,5-bis(4-hexadecyloxy-3-methoxyphenyl)-3-oxo-1,5-pentanedisulfonic acid diammonium salt (4) 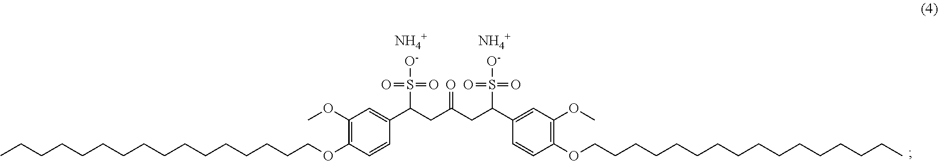 [SMILES: [NH4+] ; [NH4+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]
1,5-bis(3-methoxy-4-octadecyloxyphenyl)-3-oxo-1,5-pentanedisulfonic acid diammonium salt (5) 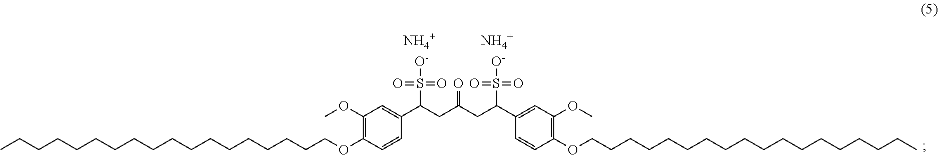 [SMILES: [NH4+] ; [NH4+] ; CCCCCCCCCCCCCCCCCCOc1ccc(C(CC(=O)CC(c2ccc(OCCCCCCCCCCCCCCCCCC)c(OC)c2)S(=O)(=O)[O-])S(=O)(=O)[O-])cc1OC]
1,5-bis(4-dodecyloxy-3-methoxyphenyl)-3-oxo-1,5-pentanedisulfonic acid disodium salt (6) 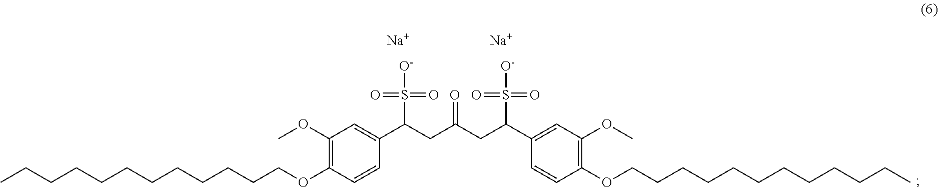 [SMILES: [Na+] ; [Na+] ; CCCCCCCCCCCCOc1ccc(C(CC(=O)CC(c2ccc(OCCCCCCCCCCCC)c(OC)c2)S(=O)(=O)[O-])S(=O)(=O)[O-])cc1OC]
1,5-bis(3-methoxy-4-tetradecyloxyphenyl)-3-oxo-1,5-pentanedisulfonic acid disodium salt (7) 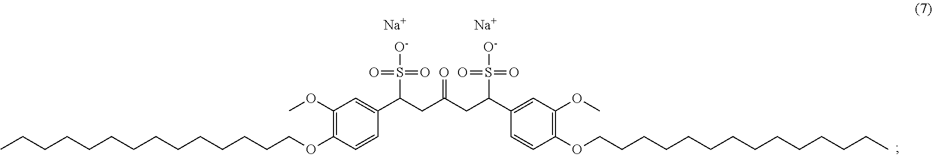 [SMILES: [Na+] ; [Na+] ; CCCCCCCCCCCCCCOc1ccc(C(CC(=O)CC(c2ccc(OCCCCCCCCCCCCCC)c(OC)c2)S(=O)(=O)[O-])S(=O)(=O)[O-])cc1OC]
1,5-bis(4-hexadecyloxy-3-methoxyphenyl)-3-oxo-1,5-pentanedisulfonic acid disodium salt (8) 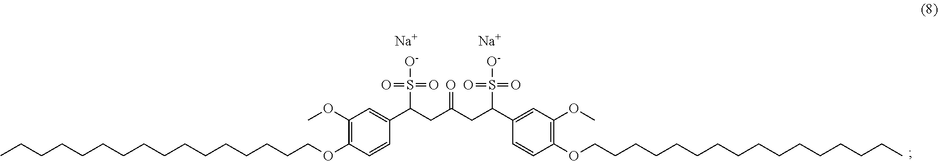 [SMILES: [Na+] ; [Na+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]
1,5-bis(3-methoxy-4-octadecyloxyphenyl)-3-oxo-1,5-pentanedisulfonic acid disodium salt (9) 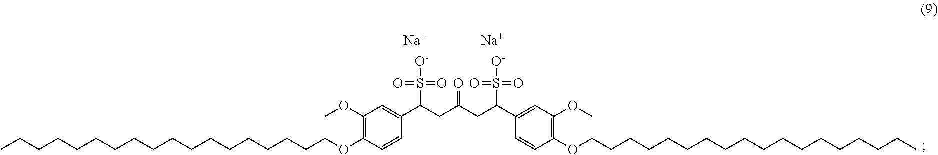 [SMILES: [Na+] ; [Na+] ; CCCCCCCCCCCCCCCCCCOc1ccc(C(CC(=O)CC(c2ccc(OCCCCCCCCCCCCCCCCCC)c(OC)c2)S(=O)(=O)[O-])S(=O)(=O)[O-])cc1OC]
1,5-bis(4-hexadecyloxy-3-methoxyphenyl)-3-oxo-1,5-pentanedisulfonic acid dipotassium salt (10) 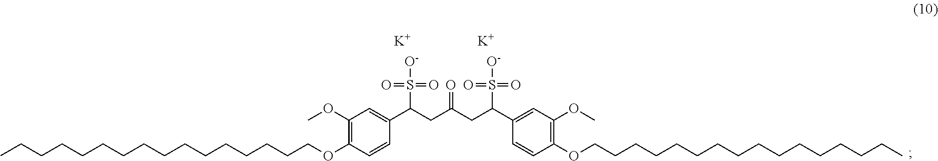 [SMILES: [K+] ; [K+] ; CCCCCCCCCCCCCCCCOc1ccc(C(CC(=O)CC(c2ccc(OCCCCCCCCCCCCCCCC)c(OC)c2)S(=O)(=O)[O-])S(=O)(=O)[O-])cc1OC]
1,5-bis(4-dodecyloxy-3-methoxyphenyl)-3-oxo-1,5-pentanedisulfonic acid dicholine salt (11) 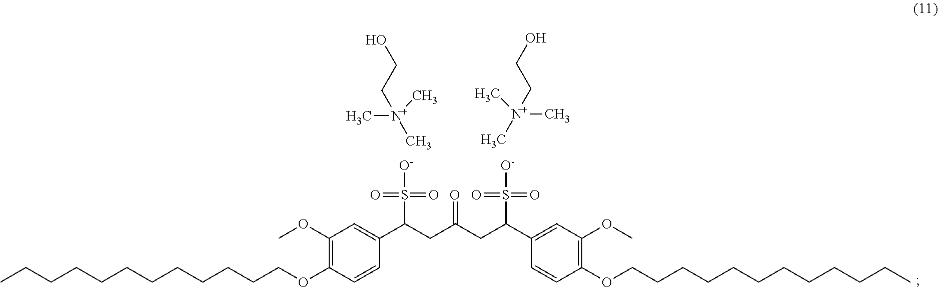
1,5-bis(4-dodecyloxy-3-methoxyphenyl)-3-oxo-1,5-pentanedisulfonic acid bis(triethanolammonium) salt (12) 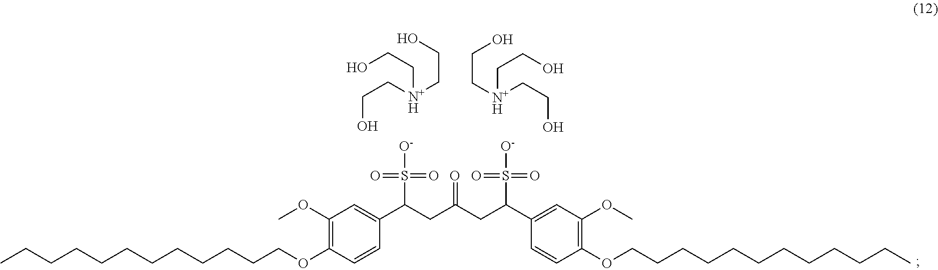
1,1′-(2-oxocyclopentane-1,3-diyl)-bis[(3-methoxy-4-octyloxyphenyl)methanesulfonic ammonium salt] (13) acid

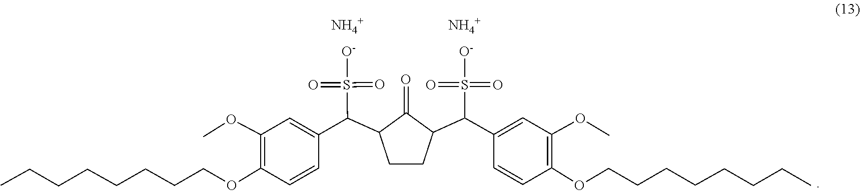 [SMILES: [NH4+] ; [NH4+] ; CCCCCCCCOc1ccc(C(C2CCC(C(c3ccc(OCCCCCCCC)c(OC)c3)S(=O)(=O)[O-])C2=O)S(=O)(=O)[O-])cc1OC]

6. A method for preparing a sulfonate compound according to claim 1 comprising the following steps:
1) reacting a 4-hydroxybenzaldehyde derivative according to the following formula (III): 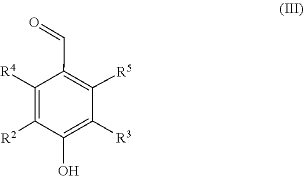
wherein
R² to R⁵ are each independently selected from hydrogen and linear, branched or cyclic hydrocarbon radicals having 1 to 26 carbon atoms, wherein optionally at least one carbon atom may be replaced by an oxygen or sulfur atom;
with a compound of the formula R¹—Y, wherein R¹ is selected from linear, branched or cyclic hydrocarbon radicals having 4 to 26 carbon atoms, wherein optionally at least one carbon atom may be replaced by an oxygen or sulfur atom, and Y represents a leaving group selected from halides and sulfonates, by means of an etherification reaction according to Williamson in the presence of a base in an organic solvent, resulting in a corresponding ether according to formula (IV): 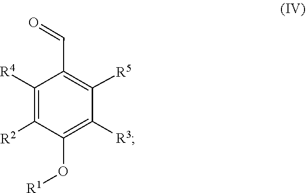
2) reacting the ether of formula (IV) with half an equivalent of acetone or an acetone derivative according to formula (V) 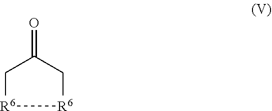
wherein each R⁶ is independently selected from hydrogen and saturated hydrocarbon radicals having 1 to 6 carbon atoms, wherein optionally the two radicals R⁶ may be connected, as indicated by the dashed line, to form a five-or six-membered ring comprising the carbonyl carbon atom, by means of a double, crossed aldol condensation reaction according to Claisen-Schmidt using an acidic or basic catalyst in an organic solvent, resulting in a corresponding unsaturated ketone according to formula (VI): 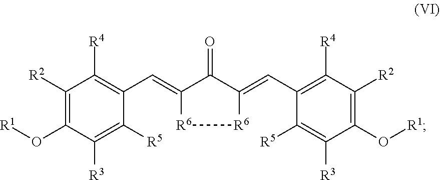
3) reacting the ketone of formula (VI) with a sulfonating agent in an alcoholic solvent for adding one or two equivalent(s) of hydrogen sulfite to the double bond(s) of the ketone of formula (VI), optionally followed by an ion exchange of the mono-or disulfonate thus obtained and introduction of predetermined counterions Xⁿ⁺, wherein n is ≥1, resulting in the sulfonate compound according to formula (I) or (II).

7. The method according to claim 6, wherein in step 1),
chloride or bromide is used as said leaving group Y; and/or
K₂CO₃ is used as said base; and/or
acetonitrile is used as said organic solvent.

8. The method according to claim 7, wherein in step 1),
bromide is used as said leaving group Y,
two equivalents of K₂CO₃ are used as said base, and
the reaction is carried out in refluxing acetonitrile.

9. The method according to claim 6, wherein in step 2),
lithium hydroxide monohydrate, LiOH.H₂O, is used as said basic catalyst; and/or
a lower alcohol, an ether, or a mixture thereof, is used as said organic solvent.

10. The method according to claim 9, wherein in step 2),
lithium hydroxide monohydrate LiOH.H₂O in an amount of 1-10 mol % is used as said basic catalyst,
isopropanol is used as said organic solvent, and
the reaction is carried out at 40-50° C.

11. The method according to claim 6, wherein in step 3),
a hydrogen sulfite or a disulfite is used as said sulfonating agent; and/or
a mixture of a lower alcohol and water is used as said alcoholic solvent; and/or
an amine is used as a catalyst.

12. The method according to claim 11, wherein in step 3),
sodium disulfite Na₂S₂O₅, calcium hydrogen sulfite Ca(HSO₃)₂, ammonium hydrogen sulfite NH₄HSO₃ or trimethylammonium sulfite [(CH₃)₃N]₂SO₃ is used as said sulfonating agent,
triethylamine, triethanolamine or choline hydroxide is used as said catalyst, and
aqueous methanol or isopropanol is used as said alcoholic solvent.

13. The method according to claim 12, wherein in step 3),
the hydrogen sulfite or disulfite is used in an amount of three equivalents of hydrogen sulfite and the amine catalyst is used an amount of at least 20 mol %, each based on the ketone of formula (VI), to obtain a disulfonate compound according to formula (I), and
refluxing aqueous isopropanol is used as said alcoholic solvent.

14. The method according to claim 6, wherein
a) in step 3), the sulfonate adduct obtained is first subjected to an ion exchange using an acidic ion exchange resin and water elution, for converting the sulfonate group(s) into the free sulfonic acid group(s), which optionally may be neutralized using an aqueous solution of the hydroxides of predetermined counterions Xⁿ⁺, to obtain the sulfonate compound according to formula (I) or (II); and/or
b) steps 2) and 3) are carried out as a one-pot synthesis.

15. A surfactant comprising a disulfonate compound of formula (I) or (II) according to claim 1, wherein the total number of carbon atoms of the radicals R¹ to R⁵ is at least 9.
